SEQUENCE LISTING (2 sequences)

SEQ ID NO: 1 - DNA, 933 bp, Arabidopsis thaliana
  misc_feature: TIGR database locus F18014_8
atgacgagcaagattctagttatcggagctacaggtctcatcggaaaagtcctcgtagaggaaagcgccaagtctggccacgccactttcgctctggttagagaagcctctctctccgatcccgttaaggcccaactcgttgagagattcaaagatctcggcgttaccatactctacggaagtttaagtgataaagagagcttagtgaaggcgattaaacaagtcgatgtcgtgatatcagctgttggtcggtttcaaactgaaattcttaatcaaaccaatatcatcgatgccatcaaagaatctggaaatgttaagagattcttaccgtcggaatttggtaatgacgtggatcgtacggtagccattgagccaacgctatcagagttcatcactaaagctcagataaggcgtgccattgaagctgcaaagataccttatacgtatgtcgtctcaggttgctttgcaggtctttttgttccttgtttgggtcaatgccacttgcgactcagatctcctcctagagacaaagtttcaatctatgatactggcaatggcaaagccattgtcaacactgaagaagacatcgttgcatatacattgaaagctgttgatgatccgagaacacttaacaagattctctacattcacccgcccaattacattgtatcgcagaacgatatggttggtttgtgggaggaaaagatcggtaagactctcgaaaagacttatgtttcagaggaagaactcctcaaaaccatccaagagagtaagcctccaatggattttttggtgggtctgatccatacaatccttgtgaagagtgactttacctccttcactatagatccttcttttggagttgaggcttccgagctttaccctgaagtcaagtacactagtgttgatgagtttcttaaccggtttatctga

SEQ ID NO: 2 - protein, 310 aa, Arabidopsis thaliana
MTSKILVIGATGLIGKVLVEESAKSGHATFALVREASLSDPVKAQLVERFKDLGVTILYGSLSDKESLVKAIKQVDVVISAVGRFQTEILNQTNIIDAIKESGNVKRFLPSEFGNDVDRTVAIEPTLSEFITKAQIRRAIEAAKIPYTYVVSGCFAGLFVPCLGQCHLRLRSPPRDKVSIYDTGNGKAIVNTEEDIVAYTLKAVDDPRTLNKILYIHPPNYIVSQNDMVGLWEEKIGKTLEKTYVSEEELLKTIQESKPPMDFLVGLIHTILVKSDFTSFTIDPSFGVEASELYPEVKYTSVDEFLNRFI

(19) United States
(12) Patent Application Publication
(10) Pub. No.: US 2002/0177527 A1
(43) Pub. Date: Nov. 28, 2002

(54) Methods for the identification of inhibitors of 2'-hydroxyisoflavone reductase expression or activity in plants

(76) Inventors: Neil Hoffman, Chapel Hill, NC (US); John W. Rice, Pittsboro, NC (US); Keith Davis, Durham, NC (US); Adel Zayed, Durham, NC (US); Robert Ascenzi, Cary, NC (US); Douglas Boyes, Chapel Hill, NC (US); Jorn Gorlach, Manchester, NJ (US); Jeffrey Woessner, Hillsborough, NC (US); Carol Hamilton, Apex, NC (US); Kenneth Phillips, Durham, NC (US)

Correspondence Address: Paradigm Genetics, Inc., Patent and Trademark Department, 108 Alexander Drive, P.O. Box 14528, Research Triangle Park, NC, 27709-4528, US

(21) Appl. No.: 10/147,761

(22) Filed: May 16, 2002

Related U.S. Application Data
(60) Provisional application No. 60/291,738, filed on May 17, 2001.

(51) Int. Cl. A01N 25/00; G01N 33/53; G01N 33/567

(52) U.S. Cl. 504/116.1; 435/7.2

(57) ABSTRACT

The present invention discloses that 2′-hydroxyisoflavone reductase (IFR) is essential for plant growth. Specifically, the inhibition of IFR gene expression in plant seedlings results in seedlings that fail to produce roots or leaves. Thus, IFR is a useful target for the identification of herbicides. Accordingly, the present invention provides compositions and methods for the identification of compounds that inhibit IFR expression or activity.

### CROSS REFERENCE TO RELATED APPLICATIONS

This application claims the benefit of U.S. Provisional Application 60/291,738 filed on May 17, 2001.

### FIELD OF THE INVENTION

The invention relates generally to plant molecular biology. In particular, the invention relates to methods for the identification of herbicides.

### BACKGROUND OF THE INVENTION

2′-hydroxyisoflavone reductase (EC 1.3.1.45) (IFR) (other names: NADPH: 2′-hydroxyisoflavone oxidoreductase; isoflavone reductase; 2′, 7-dihydroxy-4′,5′-methylenedioxyisoflavone reductase) catalyzes the reaction vestitone+NADP+=2′-hydroxyformononetin+NADPH. In the reverse direction, a 2′-hydroxyisoflavone is reduced to an isoflavonone. A second enzyme 2′-hydroxypseudobaptigenin is also involved in this reaction. 2′-hydroxyisoflavone reductase is involved in the biosynthesis of the pterocarpan phytoalexins medicarpin and maackiain. Medicarpin is synthesized by way of the isoflavonoid branch of phenylpropanoid metabolism.

Products of the phenylpropanoid pathway of secondary metabolism are involved in defense against pathogens (isoflavonoid phytoalexins).

2′-hydroxyisoflavone reductase cDNA clones have been isolated from e.g. Pisum sativum (Paiva et al. (1994) Arch Biochem Biophys 312: 501-10 (PMID: 8037464)) and Medicago sativo (Paiva et al. (1991) Plant Mol Biol 17: 653-67 (PMID: 1912490)). The enzyme was isolated from chickpea cell cultures by Tiemann et al. ((1991) Eur J Biochem 200: 751-7 (PMID: 1915347)).

The present invention discloses the essentiality of 2′-hydroxyisoflavone reductase for plant growth and developent and, thus, its potential as a herbicide target. The production of effective new herbicides is increasingly important as the use of herbicides to control undesirable vegetation such as weeds in crop fields has become an almost universal practice. The herbicide market exceeds 15 billion dollars annually. Despite this extensive use, weed control remains a significant and costly problem for farmers. Effective use of herbicides requires sound management, and various weed species are resistant to the existing herbicides. For these reasons, the identification of new herbicides is highly desirable. The present invention provides methods for the identification of inhibitors of 2′-hydroxyisoflavone reductase activity for use as herbicides.

### SUMMARY OF THE INVENTION

The present invention discloses that antisense expression of an IFR cDNA in Arabidopsis causes severe developmental abnormalities such as plant seedlings that fail to produce roots or leaves. Thus, the present inventors have discovered that IFR is essential for normal seed development and growth and is useful as a target for the identification of herbicides. Accordingly, the present invention provides compositions and methods for the identification of compounds that inhibit IFR expression or activity.

### BRIEF DESCRIPTION OF THE DRAWINGS

FIG. 1 shows the 2′-Hydroxyisoflavone reductase reaction.

FIG. 2 is a digital image showing the effect of IFR antisense expression on Arabidopsis thaliana seeds and seedlings (PPG227/S5569).

FIG. 3 is a digital image showing the effect of IFR antisense expression on Arabidopsis thaliana seeds and seedlings (PPG1073/S21057).

### DETAILED DESCRIPTION OF THE INVENTION

For clarity, certain terms used herein are presented as follows:

The term “binding” refers to a noncovalent interaction that holds two molecules together. For example, two such molecules could be an enzyme and an inhibitor of that enzyme. Noncovalent interactions include hydrogen bonding, ionic interactions among charged groups, van der Waals interactions and hydrophobic interactions among nonpolar groups. One or more of these interactions can mediate the binding of two molecules to each other.

A “conservative amino acid substitution” is for example an amino acid substitution where the substituted amino acid residue has similar chemical properties (e.g. charge or hydrophobicity) to the reference amino acid residue. In general, a substitution of an amino acid for another amino acid having the same type of R group is considered a conservative substitution. Amino acids can be classified into the following R groups: nonpolar aliphatic; polar uncharged; positively charged; negatively charged; and aromatic. Glycine, alanine, valine, leucine, isoleucine and proline have nonpolar aliphatic R groups. Serine, threonine, cysteine, methionine, asparagine and glutamine have polar uncharged R groups. Lysine, arginine and histidine have positively charged R groups. Aspartate and glutamate have negatively charged R groups. Phenylalanine, tyrosine and tryptophan have aromatic R groups. The phrases “percent sequence conservation” and “percent sequence similarity” are herein used interchangeably.

The phrase “a polynucleotide consisting essentially of the nucleotide sequence of SEQ ID NO: 1 ” is specifically intended to include polynucleotides that encode the polypeptide of SEQ ID NO:2 where the nucleotides of SEQ ID NO: 1 have been modified to optimize expression in a particular cell type.

The phrase “a polypeptide consisting essentially of the amino acid sequence of SEQ ID NO:2” is referring to a polypeptide having essentially the same structure and activity as the polypeptide of SEQ ID NO:2, e.g. where only substitutions in amino acids of SEQ ID NO:2 not significantly affecting the enzymatic activity are present. By “not significantly affecting the enzymatic activity of the polypeptide of SEQ ID NO:2” is meant an alteration in activity of less than 50% of the activity of the polypeptide of SEQ ID NO:2, determined according to the methods described herein. An example of a polypeptide consisting essentially of the polypeptide of SEQ ID NO:2 is a polypeptide that contains one or more substitutions in the amino acid sequence of SEQ ID NO:2, but retains at least 50% of the IFR activity of SEQ ID NO:2. Preferably the polypeptide consisting essentially of the amino acid sequence of SEQ ID NO:2 contains five or fewer conservative amino acid substitutions of SEQ ID NO:2 at positions of low sequence conservation, i.e. positions of low sequence identity between various species of IFR when aligned according to the procedures described herein.

As used herein, the term “GUS” means β-glucouronidase.

As used herein “2′-hydroxyformononetin” refers to the compound 2′-hydroxyformononetin designated by ChemACX and Chemfinder.com ACX Number X1037083-7.

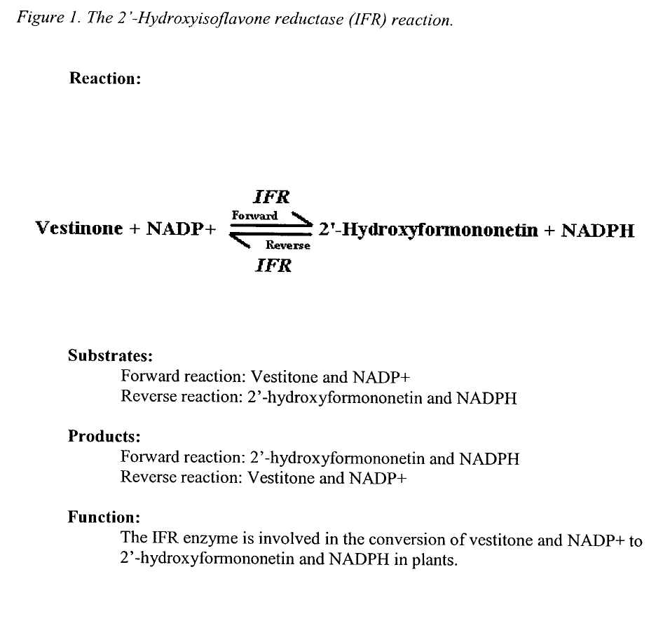

As used herein, the term “2′-Hydroxyisoflavone reductase (EC 1.3.1.45)” is synonymous with “IFR” and refers to an enzyme that catalyses the reversible conversion of vestitone and NADP+to 2′-hydroxyformononetin and NADPH, as shown in FIG. 1.

As used herein, the term “dI” means deionized.

The term “herbicide”, as used herein, refers to a compound that may be used to kill or suppress the growth of at least one plant, plant cell, plant tissue or seed.

The term “inhibitor”, as used herein, refers to a chemical substance that reduces or eliminates the enzymatic activity of IFR. The inhibitor may function by interacting directly with the enzyme, a cofactor of the enzyme, the substrate of the enzyme, or any combination thereof. By reducing the enzymatic activity of IFR is meant that the amount of a product of the enzymatic reaction is reduced by more than the margin of error inherent in the measurement technique of the invention. The reduction is a decrease of at least about 20% or greater of the activity of the enzyme in the absence of the inhibitor, more desirably a decrease by about 25%, 30%, 35%, 40%, 45%, 50%, 55%, 60%, 65%, 70%, 75%, 80%, 85%, 90%, 95%, 98%, 99%, or greater. Another example of an inhibitor is a compound that causes abnormal growth of a host cell by interacting with the gene product encoded by the nucleotide sequence of the present invention.

A polynucleotide may be “introduced” into a plant cell by any means, including transfection, transformation or transduction, electroporation, particle bombardment, agroinfection and the like. The introduced polynucleotide may be maintained in the cell stably if it is incorporated into a non-chromosomal autonomous replicon or integrated into the plant chromosome. Alternatively, the introduced polynucleotide may be present on an extra-chromosomal non-replicating vector and be transiently expressed or transiently active.

As used herein, the term “Ni-NTA” refers to nickel sepharose.

As used herein, the term “PGI” means plant growth inhibition.

The “percent (%) sequence identity” between two polynucleotide or two polypeptide sequences is determined according to the either the BLAST program (Basic Local Alignment Search Tool; Altschul and Gish (1996) Meth Enzymol 266:460-480 and Altschul (1990) J Mol Biol 215:403-410) in the Wisconsin Genetics Software Package (Devererreux et al. (1984) Nucl Acid Res 12:387), Genetics Computer Group (GCG), Madison, Wis. (NCBI, Version 2.0.11, default settings) or using Smith Waterman Alignment (Smith and Waterman (1981) Adv Appl Math 2:482) as incorporated into GeneMatcher Plus™ ( using the default settings and the version current at the time of filing). It is understood that for the purposes of determining sequence identity when comparing a DNA sequence to an RNA sequence, a thymine nucleotide is equivalent to a uracil nucleotide.

“Plant” refers to whole plants, plant organs and tissues (e.g., stems, roots, ovules, stamens, leaves, embryos, meristematic regions, callus tissue, gametophytes, sporophytes, pollen, microspores and the like) seeds, plant cells and the progeny thereof.

By “polypeptide” is meant a chain of at least four amino acids joined by peptide bonds. The chain may be linear, branched, circular or combinations thereof. The polypeptides may contain amino acid analogs and other modifications, including, but not limited to glycosylated or phosphorylated residues.

As used herein in “vestitone” refers to the compound vestitone designated by Chemical Abstracts Service Registry Number 57462-46-1.

### Embodiments of the Invention

The present inventors have discovered that inhibition of IFR gene expression strongly inhibits the growth and development of plant seedlings. Thus, the inventors are the first to demonstrate that IFR is a target for herbicides.

Accordingly, the invention provides methods for identifying compounds that inhibit IFR gene expression or activity. Such methods include ligand binding assays, assays for enzyme activity and assays for IFR gene expression. Any compound that is a ligand for IFR, other than its substrates for the forward and reverse reactions, vestitone, 2′-hydroxyformononetin, NADP+, and NADPH, may have herbicidal activity. For the purposes of the invention, “ligand” refers to a molecule that will bind to a site on a polypeptide. The compounds identified by the methods of the invention are useful as herbicides.

Thus, in one embodiment, the invention provides a method for identifying a compound as a candidate for a herbicide. The method comprises contacting an IFR with a compound and detecting the presence and/or absence of binding between said compound and said IFR, wherein binding indicates that said compound is a candidate for a herbicide.

By “IFR” is meant any enzyme that catalyzes the interconversion of vestitone and NADP+ with 2′-hydroxyformononetin and NADPH. The IFR may have the amino acid sequence of a naturally occurring IFR found in a plant, animal or microorganism, such as SEQ ID NO:2. The IFR polypeptides of the invention may also consist essentially of the amino acid sequence of SEQ ID NO:2, e.g. where only substitutions in amino acids of SEQ ID NO:2 not significantly affecting the enzymatic activity are present. An example of a polypeptide consisting essentially of the polypeptide of SEQ ID NO:2 is a polypeptide that contains one or more substitutions in the amino acid sequence of SEQ ID NO:2, but retains at least 50% of the IFR activity of SEQ ID NO:2. Preferably the polypeptide consisting essentially of the amino acid sequence of SEQ ID NO:2 contains five or fewer conservative amino acid substitutions of SEQ ID NO:2 at positions of low sequence conservation, i.e. positions of low sequence identity between various species of IFR when aligned generally according to the procedures described herein and known to those of skill in the art.

Preferably the IFR is a plant IFR. The cDNA (SEQ ID NO:1) encoding the IFR protein or polypeptide (SEQ ID NO:2) can be found herein as well as in the TIGR database at locus F18014_{—}8. The invention is specifically intended to also include polynucleotides consisting essentially of the nucleotide sequence of SEQ ID NO:1 wherein said polynucleotides encode the polypeptide of SEQ ID NO:2 but have been modified to optimize expression in a particular cell type. By “plant IFR” is meant an enzyme that can be found in at least one plant, and which catalyzes the interconversion of vestitone and NADP+ with 2′-hydroxyformononetin and NADPH. The IFR may be from any plant, including both monocots and dicots.

In one embodiment, the IFR is an Arabidopsis IFR. Arabidopsis species include, but are not limited to, Arabidopsis arenosa, Arabidopsis bursifolia, Arabidopsis cebennensis, Arabidopsis croatica, Arabidopsis griffithiana, Arabidopsis halleri, Arabidopsis himalaica, Arabidopsis korshinskyi, Arabidopsis lyrata, Arabidopsis neglecta, Arabidopsis pumila, Arabidopsis suecica, Arabidopsis thaliana and Arabidopsis wallichii. Preferably, the Arabidopsis IFR is from Arabidopsis thaliana.

In various embodiments, the IFR can be from barnyard grass (Echinochloa crus-galli), crabgrass (Digitaria sanguinalis), green foxtail (Setana viridis), perennial ryegrass (Lolium perenne), hairy beggarticks (Bidens pilosa), nightshade (Solanum nigrum), smartweed (Polygonum lapathifolium), velvetleaf (Abutilon theophrasti), common lambsquarters (Chenopodium album L.), Brachiara plantaginea, Cassia occidentalis, Ipomoea aristolochiaefolia, Ipomoea purpurea, Euphorbia heterophylla, Setaria spp, Amaranthus retroflexus, Sida spinosa, Xanthium strumarium and the like.

Fragments of an IFR polypeptide may be used in the methods of the invention. The fragments comprise at least 10 consecutive amino acids of an IFR. Preferably, the fragment comprises at least 15, 20, 25, 30, 35, 40, 50, 60, 70, 80, 90, 100, 150, 200, 250, 275, 280, 285, 290, 295, 300, 305, 306, 307, 308, 309, or 310 consecutive amino acids residues of an IFR, up to the entire length of an IFR. In one embodiment, the fragment is from an Arabidopsis IFR. Preferably, the fragment contains an amino acid sequence conserved among plant 2′-Hydroxyisoflavone reductases. Such conserved fragments are identified in Grima-Pettenuti et al. (1993) Plant Mol Biol 21:1085-1095 and Taveres et al. (2000), supra. Those skilled in the art could identify additional conserved fragments using sequence comparison software.

Polypeptides having at least 80% sequence identity with SEQ ID NO:2 are also useful in the methods of the invention. Preferably, the sequence identity is at least 85%, more preferably the identity is at least 90%, 91%, 92%, 93%, 94%, 95%, 96%, 97%, 98%, or 99%. In addition, it is preferred that the polypeptide has at least 50% of the activity of the plant IFR of SEQ ID NO:2. More preferably, the polypeptide has at least 60%, at least 70%, at least 75%, 80%, 85%, 90%, 92%, 95%, 97%, or at least 99% of the activity of SEQ ID NO:2.

Thus, in another embodiment, the invention provides a method for identifying a compound as a candidate for a herbicide, comprising contacting the compound with at least one polypeptide selected from the group consisting of a plant IFR, a polypeptide consisting essentially of SEQ ID NO:2, a polypeptide comprising a fragment of a plant IFR, a polypeptide comprising a fragment of SEQ ID NO:2, a polypeptide having at least 85% sequence identity with SEQ ID NO:2, and a polypeptide having at least 85% sequence identity with SEQ ID NO:2 and at least 50% of the activity thereof. The method further comprises detecting the presence and/or absence of binding between the compound and the polypeptide, wherein binding indicates that the compound is a candidate for a herbicide.

Any technique for detecting the binding of a ligand to its target may be used in the methods of the invention. For example, the ligand and target are combined in a buffer. Many methods for detecting the binding of a ligand to its target are known in the art, and include, but are not limited to the detection of an immobilized ligand-target complex or the detection of a change in the properties of a target when it is bound to a ligand. For example, in one embodiment, an array of immobilized candidate ligands is provided. The immobilized ligands are contacted with an IFR protein or a fragment or variant thereof, the unbound protein is removed and the bound IFR is detected. In a preferred embodiment, bound IFR is detected using a labeled binding partner, such as a labeled antibody. In a variation of this assay, IFR is labeled prior to contacting the immobilized candidate ligands. Preferred labels include fluorescent or radioactive moieties. Preferred detection methods include fluorescence correlation spectroscopy (FCS) and FCS-related confocal nanofluorimetric methods.

Once a compound is identified as a candidate for a herbicide, it can be tested for the ability to inhibit IFR enzyme activity. The compounds can be tested using either in vitro or cell based enzyme assays. Alternatively, a compound can be tested by applying it directly to a plant or plant cell, or expressing it therein, and monitoring the plant or plant cell for changes or decreases in growth, development, viability or alterations in gene expression.

Thus, in one embodiment, the invention provides a method for determining whether a compound identified as a herbicide candidate by an above method has herbicidal activity, comprising: contacting a plant or plant cells with said herbicide candidate and detecting the presence or absence of a decrease in the growth or viability of said plant or plant cells.

By decrease in growth, is meant that the herbicide candidate causes at least a 10% decrease in the growth of the plant or plant cells, as compared to the growth of the plants or plant cells in the absence of the herbicide candidate. By a decrease in viability is meant that at least 20% of the plants cells, or portion of the plant contacted with the herbicide candidate are nonviable. Preferably, the growth or viability will be at decreased by at least 40%. More preferably, the growth or viability will be decreased by at least 50%, 75% or at least 90% or more. Methods for measuring plant growth and cell viability are known to those skilled in the art. It is possible that a candidate compound may have herbicidal activity only for certain plants or certain plant species.

The ability of a compound to inhibit IFR activity can be detected using in vitro enzymatic assays in which the disappearance of a substrate or the appearance of a product is directly or indirectly detected. IFR catalyzes the irreversible or reversible reaction of vestitone and NADP+ to 2′-hydroxyformononetin and NADPH. Methods for detection of vestitone, NADP+, 2′-hydroxyformononetin, and/or NADPH, include spectrophotometry, mass spectroscopy, thin layer chromatography (TLC) and reverse phase HPLC.

Thus, the invention provides, for the forward reaction, a method for identifying a compound as a candidate for a herbicide, comprising contacting a vestitone and NADP+ with IFR, contacting said vestitone and NADP+ with IFR and said candidate compound and, determining the concentration of 2′-hydroxyfornononetin and/or NADPH after the contacting of steps (a) and (b). If a candidate compound inhibits IFR activity, a higher concentration of the substrates (vestitone and NADP+) and a lower level of the products (2′-hydroxyformononetin and NADPH) will be detected in the presence of the candidate compound (step b) than in the absence of the compound (step a).

And, the invention provides for the reverse reaction, a method for identifying a compound as a candidate for a herbicide, comprising contacting a 2′-hydroxyformononetin and NADPH with IFR, contacting said 2′-hydroxyformononetin and NADPH with IFR and said candidate compound, and determining the concentration of vestitone and/or NADP+ after the contacting of steps (a) and (b). If a candidate compound inhibits IFR activity, a higher concentration of the substrates (2′-hydroxyformononetin and NADPH) and a lower level of the products (vestitone and NADP+) will be detected in the presence of the candidate compound (step b) than in the absence of the compound (step a).

Preferably the IFR is a plant IFR. Enzymatically active fragments of a plant IFR are also useful in the methods of the invention. For example, a polypeptide comprising at least 100 consecutive amino acid residues of SEQ ID NO:2 is used in the methods of the invention. In addition, a polypeptide having at least 80%, 85%, 90%, 95%, 98% or at least 99% sequence identity with SEQ ID NO:2 is used in the methods of the invention. Preferably, the polypeptide has at least 80% sequence identity with SEQ ID NO:2 and at least 50%, 60%, 75%, 80%, 85%, 90% or at least 95% of the activity thereof.

Thus, the invention provides, for the forward reaction, a method for identifying a compound as a candidate for a herbicide, comprising contacting vestitone and NADP+ with a polypeptide selected from the group consisting of a polypeptide having at least 85% sequence identity with SEQ ID NO:2, a polypeptide having at least 80% sequence identity with SEQ ID NO:2 and at least 50% of the activity thereof, and a polypeptide comprising at least 100 consecutive amino acids of SEQ ID NO:2. The method further comprises contacting said vestitone and NADP+ with said polypeptide and said compound, and determining the concentration of 2′-hydroxyformononetin and/or NADPH after the contacting of steps (a) and (b). Again, if a candidate compound inhibits IFR activity, a higher concentration of the substrates (vestitone and NADP+) and a lower level of the products (2′-hydroxyformononetin and NADPH) will be detected in the presence of the candidate compound (step b) than in the absence of the compound (step a).

And, the invention provides, for the reverse reaction, a method for identifying a compound as a candidate for a herbicide, comprising contacting a 2′-hydroxyformononetin and NADPH with a polypeptide selected from the group consisting of a polypeptide having at least 85% sequence identity with SEQ ID NO:2, a polypeptide having at least 80% sequence identity with SEQ ID NO:2 and at least 50% of the activity thereof, and a polypeptide comprising at least 100 consecutive amino acids of SEQ ID NO:2. The method further comprising contacting said 2′-hydroxyformononetin and NADPH with said polypeptide and said compound, and determining the concentration of vestitone and/or NADP+ after the contacting of steps (a) and (b). Again, if a candidate compound inhibits IFR activity, a higher concentration of the substrates (2′-hydroxyformononetin and NADPH) and a lower level of the products (vestitone and NADP+) will be detected in the presence of the candidate compound (step b) than in the absence of the compound (step a).

For the in vitro enzymatic assays, IFR protein and derivatives thereof may be purified from a plant or may be recombinantly produced in and purified from a plant, bacteria, or eukaryotic cell culture. Preferably these proteins are produced using a baculovirus or E. coli expression system. Methods for the purification of 2′-hydroxyisoflavone reductase are described in Tiemann K et al. (1991) “Purification, characterization and CDNA cloning of NADPH:isoflavone oxidoreductase.” Eur J Biochem 200: 751-7 (PMID: 1915347) and/or Paiva et al. (1991) Stress responses in alfalfa (Medicago sativa L.) 11. “Molecular cloning and expression of alfalfa isoflavone reductase, a key enzyme of isoflavonoid phytoalexin biosynthesis.” Plant Mol Biol 17: 653-67 (PMID: 1912490). Other methods for the purification of IFR proteins and polypeptides are known to those skilled in the art.

As an alternative to in vitro assays, the invention also provides plant and plant cell based assays. In one embodiment, the invention provides a method for identifying a compound as a candidate for a herbicide, comprising measuring the expression of IFR in a plant or plant cell in the absence of said compound, contacting a plant or plant cell with said compound and measuring the expression of IFR in said plant or plant cell, and comparing the expression of IFR in steps (a) and (b). A reduction in IFR expression indicates that the compound is a herbicide candidate. In one embodiment, the plant or plant cell is an Arabidopsis thaliana plant or plant cell.

Expression of IFR can be measured by detecting IFR primary transcript or mRNA, IFR polypeptide or IFR enzymatic activity. Methods for detecting the expression of RNA and proteins are known to those skilled in the art. See, for example, Current Protocols in Molecular Biology Ausubel et al., eds., Greene Publishing and Wiley-Interscience, New York, 1995. The method of detection is not critical to the invention. Methods for detecting IFR RNA include, but are not limited to amplification assays such as quantitative PCR, and/or hybridization assays such as Northern analysis, dot blots, slot blots, in-situ hybridization, transcriptional fusions using an IFR promoter fused to a reporter gene, bDNA assays and microarray assays.

Methods for detecting protein expression include, but are not limited to, immunodetection methods such as Western blots, His Tag and ELISA assays, polyacrylamide gel electrophoresis, mass spectroscopy and enzymatic assays. Also, any reporter gene system may be used to detect IFR protein expression. For detection using gene reporter systems, a polynucleotide encoding a reporter protein is fused in frame with IFR, so as to produce a chimeric polypeptide. Methods for using reporter systems are known to those skilled in the art. Examples of reporter genes include, but are not limited to, chloramphenicol acetyltransferase (Gorman et al. (1982) Mol Cell Biol 2:1104; Prost et al. (1986) Gene 45:107- 111), β-galactosidase (Nolan et al (1988) Proc Natl Acad Sci USA 85:2603-2607), alkaline phosphatase (Berger et al. (1988) Gene 66:10), luciferase (De Wet et aL (1987) Mol Cell Biol 7:725-737), β-glucuronidase (GUS), fluorescent proteins, chromogenic proteins and the like. Methods for detecting IFR activity are described above.

Chemicals, compounds or compositions identified by the above methods as modulators of IFR expression or activity can then be used to control plant growth. For example, compounds that inhibit plant growth can be applied to a plant or expressed in a plant, in order to prevent plant growth. Thus, the invention provides a method for inhibiting plant growth, comprising contacting a plant with a compound identified by the methods of the invention as having herbicidal activity.

Herbicides and herbicide candidates identified by the methods of the invention can be used to control the growth of undesired plants, including both monocots and dicots. Examples of undesired plants include, but are not limited to barnyard grass (Echinochloa crus-galli), crabgrass (Digitaria sanguinalis), green foxtail (Setana viridis), perennial ryegrass (Lolium perenne), hairy beggarticks (Bidens pilosa), nightshade (Solanum nigrum), smartweed (Polygonum lapathifolium), velvetleaf (Abutilon theophrasti), common lambsquarters (Chenopodium album L.), Brachiara plantaginea, Cassia occidentalis, Ipomoea aristolochiaefolia, Ipomoea purpurea, Euphorbia heterophylla, Setaria spp, Amaranthus retroflexus, Sida spinosa, Xanthium strumarium and the like.

### EXPERIMENTAL

Plant Growth Conditions

Unless, otherwise indicated, all plants are grown Scotts Metro-Mix™ soil (the Scotts Company) or a similar soil mixture in an environmental growth room at 22° C., 65% humidity, 65% humidity and a light intensity of ˜100μ-Ε m⁻² s³¹ supplied over 16 hour day period.

Seed Sterilization

All seeds are surface sterilized before sowing onto phytagel plates using the following protocol.

1. Place approximately 20-30 seeds into a labeled 1.5 ml conical screw cap tube. Perform all remaining steps in a sterile hood using sterile technique.

2. Fill each tube with 1 ml 70% ethanol and place on rotisserie for 5 minutes.

3. Carefully remove ethanol from each tube using a sterile plastic dropper; avoid removing any seeds.

4. Fill each tube with 1 ml of 30% Clorox and 0.5% SDS solution and place on rotisserie for 10 minutes.

5. Carefully remove bleach/SDS solution.

6. Fill each tube with 1 ml sterile dI H₂O; seeds should be stirred up by pipetting of water into tube. Carefully remove water. Repeat 3 to 5 times to ensure removal of Clorox/SDS solution.

7. Fill each tube with enough sterile dl H₂O for seed plating (˜200-400 μl). Cap tube until ready to begin seed plating.

Plate Growth Assays

Surface sterilized seeds are sown onto plate containing 40 ml half strength sterile MS (Murashige and Skoog, no sucrose) medium and 1% Phytagel using the following protocol:

1. Using pipette man and 200 μl tip, carefully fill tip with seed solution. Place 10 seeds across the top of the plate, about ¼ in down from the top edge of the plate.

2. Place plate lid ¾ of the way over the plate and allow to dry for 10 minutes.

3. Using sterile micropore tape, seal the edge of the plate where the top and bottom meet.

4. Place plates stored in a vertical rack in the dark at 4° C. for three days.

5. Three days after sowing, the plates transferred into a growth chamber with a day and night temperature of 22 and 20° C., respectively, 65% humidity and a light intensity of ˜100 μ-Ε m⁻² s⁻¹ supplied over 16 hour day period.

6. Beginning on day 3, daily measurements are carried out to track the seedlings development until day 14. Seedlings are harvested on day 14 (or when root length reaches 6 cm) for root and rosette analysis.

### EXAMPLE 1

### Construction of a Transgenic Plant expressing the Driver

The “Driver” is an artificial transcription factor comprising a chimera of the DNA-binding domain of the yeast GAL4 protein (amino acid residues 147) fused to two tandem activation domains of herpes simplex virus protein VP16 (amino acid residues 413-490). Schwechheimer et al. (1998) Plant Mol Biol 36:195-204. This chimeric driver is a transcriptional activator specific for promoters having GAL4 binding sites. Expression of the driver is controlled by two tandem copies of the constitutive CaMV 35S promoter.

The driver expression cassette was introduced into Arabidopsis thaliana by agroinfection. Transgenic plants that stably expressed the driver transcription factor were obtained.

### EXAMPLE 2

### Construction of Antisense Expression Cassettes in a Binary Vector

A fragment, fragment or variant of an Arabidopsis thaliana cDNA corresponding to SEQ ID NO:1 was ligated into the PacI/AscI sites of an E.coli/Agrobacterium binary vector in the antisense orientation. This placed transcription of the antisense RNA under the control of an artificial promoter that is active only in the presence of the driver transcription factor described above. The artificial promoter contains four contiguous binding sites for the GAL4 transcriptional activator upstream of a minimal promoter comprising a TATA box.

The ligated DNA was transformed into E. coli. Kanamycin resistant clones were selected and purified. DNA was isolated from each clone and characterized by PCR and sequence analysis. The DNA was inserted in a vector that expresses the A. thaliana antisense RNA, which is complementary to a portion of the DNA of SEQ ID NO:1. This antisense RNA is complementary to the cDNA sequence found in the TIGR database at locus F18014₁₃8. The coding sequence for this locus is shown as SEQ ID NO:1. The protein encoded by these mRNAs is shown as SEQ ID NO:2.

The antisense expression cassette and a constitutive chemical resistance expression cassette are located between right and left T-DNA borders. Thus, the antisense expression cassettes can be transferred into a recipient plant cell by agroinfection.

### EXAMPLE 3

### Transformation of Agrobacterium with the Antisense Expression Cassette

The vector was transformed into Agrobacterium tumefaciens by electroporation. Transformed Agrobacterium colonies were isolated using chemical selection. DNA was prepared from purified resistant colonies and the inserts were amplified by PCR and sequenced to confirm sequence and orientation.

### EXAMPLE 4

### Construction of an Arabidopsis Antisense Target Plants

The antisense expression cassette was introduced into Arabidopsis thaliana wild-type plants by the following method. Five days prior to agroinfection, the primary inflorescence of Arabidopsis thaliana plants grown in 2.5 inch pots were clipped in order enhance the emergence of secondary bolts.

At two days prior to agroinfection, 5 ml LB broth (10 g/L Peptone, 5 g/L Yeast extract, 5 g/L NaCl, pH 7.0 plus 25 mg/L kanamycin added prior to use) was inoculated with a clonal glycerol stock of Agrobacterium carrying the desired DNA. The cultures were incubated overnight at 28° C. at 250 rpm until the cells reached stationary phase. The following morning, 200 ml LB in a 500 ml flask was inoculated with 500 μl of the overnight culture and the cells were grown to stationary phase by overnight incubation at 28° C. at 250 rpm. The cells were pelleted by centrifugation at 8000 rpm for 5 minutes. The supernatant was removed and excess media was removed by setting the centrifuge bottles upside down on a paper towel for several minutes. The cells were then resuspended in 500 ml infiltration medium (autoclaved 5% sucrose) and 250 μl/L Silwet L-77™ (84% polyalkyleneoxide modified heptamethyltrisiloxane and 16% allyloxypolyethyleneglycol methyl ether), and transferred to a one liter beaker.

The previously clipped Arabidopsis plants were dipped into the Agrobacterium suspension so that all above ground parts were immersed and agitated gently for 10 seconds. The dipped plants were then covered with a tall clear plastic dome in order to maintain the humidity, and returned to the growth room. The following day, the dome was removed and the plants were grown under normal light conditions until mature seeds were produced. Mature seeds were collected and stored desiccated at 4° C.

Transgenic Arabidopsis T1 seedlings were selected. Approximately 70 mg seeds from an agrotransformed plant were mixed approximately 4:1 with sand and placed in a 2 ml screw cap cryo vial.

One vial of seeds was then sown in a cell of an 8 cell flat. The flat was covered with a dome, stored at 4° C. for 3 days, and then transferred to a growth room. The domes were removed when the seedlings first emerged. After the emergence of the first primary leaves, the flat was sprayed uniformly with a herbicide corresponding to the chemical resistance marker plus 0.005% Silwet (50 μl/L) until the leaves were completely wetted. The spraying was repeated for the following two days.

Ten days after the first spraying resistant plants were transplanted to 2.5 inch round pots containing moistened sterile potting soil. The transplants were then sprayed with herbicide and returned to the growth room. These herbicide resistant plants represented stably transformed T1 plants.

### Example 5

### Effect of Antisense Expression in Arabidopsis Seedlings

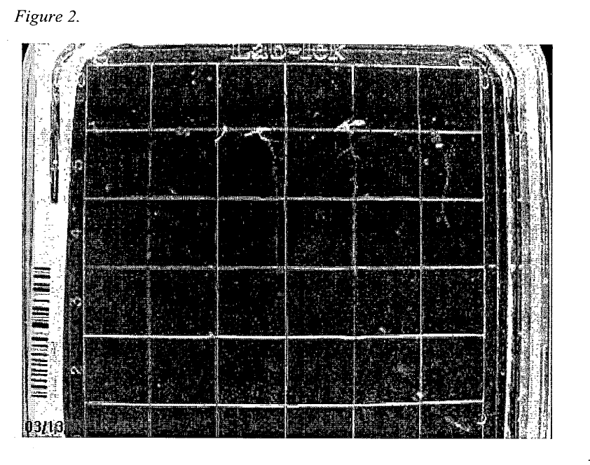

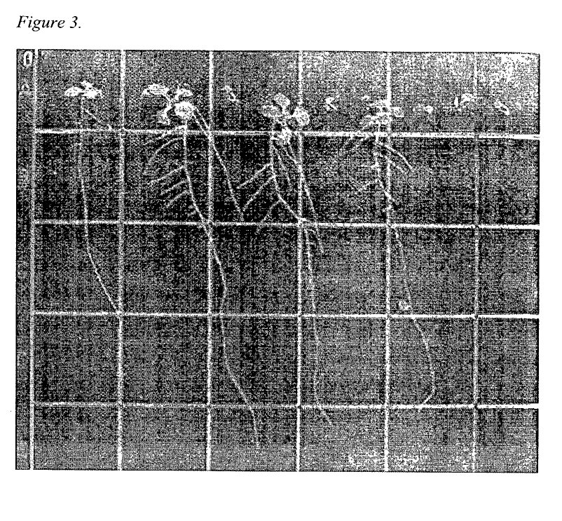

The T1 antisense target plants from the transformed plant lines obtained in Example 4 were crossed with the Arabidopsis transgenic driver line described above. The resulting F1 seeds were then subjected to a PGI plate assay to observe seedling growth over a 2-week period. Seedlings were inspected for growth and development. The transgenic plant line containing the antisense construct exhibited significant developmental abnormalities during early development. FIG. 2 and 3 show the effects of antisense expression on Arabidopsis seedlings.

The clear 1:1 segregation ratio observed in the two antisense lines demonstrates that the antisense expression of this gene resulted in significantly impaired growth and that this gene represents an essential gene for normal plant growth and development. The transgenic lines containing the antisense construct for 2′-Hydroxyisoflavone reductase exhibited significant seedling abnormalities. Seedlings did not produce any roots or leaves, as show in FIGS. 2 and 3.

### EXAMPLE 6

### Cloning & Expression Strategies, Extraction and Purfication of the IFR Protein

The following protocol can be employed to obtain purified IFR protein.

Cloning & Expression strategies:

IFR gene is cloned into E. coil (pET vectors-Novagen), Baculovirus (Pharmingen) and Yeast (Invitrogen) expression vectors containing His/fusion protein tags. The expression of recombinant protein is evaluated by SDS-PAGE and Western blot analysis.

Extraction:

Extract recombinant protein from 250 ml cell pellet in 3 mL of extraction buffer By sonicating 6 times, with 6 sec pulses at 4° C. Centrifuge extract at 15000×g for 10 min and collect supernatant. Assess biological activity of the recombinant protein by activity assay.

Purification:

Purify recombinant protein by Ni-NTA affinity chromatography (Qiagen).

Purification protocol: perform all steps at 4oC:

Use 3 ml Ni-beads (Qiagen)

Equilibrate column with the buffer

Load protein extract

Wash with the equilibration buffer

Elute bound protein with 0.5 M imidazole

### EXAMPLE 7

### Assays for Testing Inhibitors or Candidates for Inhibition of IFR Activity

The enzymatic activity of IFR is determined in the presence and absence of candidate inhibitors in a suitable reaction mixture, such as described by any of the following known assay protocols:

A. Radiochemical assay:

This assay is based on the interconversion of [¹⁴C] vestitone and [¹⁴C] 2′-hydroxyformononetin.

B. NADP+/NADPH assay:

The enzymatic activity of this enzyme is monitored by the change in absorbance at 340 nm or change in fluorescence at excitation wavelength 340 nm and emission wavelength 460 nm due to the formation of NADPH by the forward reaction. As an alternative, the loss of NADPH is monitored for the reverse reaction, as may be described in Tiemann et al. ((1991) Eur J Biochem 200: 751-7 (PMID: 1915347)).

While the foregoing describes certain embodiments of the invention, it will be understood by those skilled in the art that variations and modifications may be made and still fall within the scope of the invention.

What is claimed is:

1. A method for identifying a compound as a candidate for a herbicide, comprising:
a) contacting a plant IFR with said compound; and
b) detecting the presence and/or absence of binding between said compound and said IFR;
wherein binding indicates that said compound is a candidate for a herbicide.

2. The method of claim 1, wherein said IFR is an Arabidopsis IFR.

3. The method of claim 2, wherein said IFR is SEQ ID NO:2.

4. The method of claim 3, wherein said IFR consists essentially of SEQ ID NO:2.

5. A method for determining whether a compound identified as a herbicide candidate by the method of claim 1 has herbicidal activity, comprising: contacting a plant or plant cells with said herbicide candidate and detecting the presence or absence of a decrease in growth or viability of said plant or plant cells.

6. A method for identifying a compound as a candidate for a herbicide, comprising:
a) contacting a compound with at least one polypeptide selected from the group consisting of: an amino acid sequence comprising at least ten consecutive amino acids of a plant IFR, an amino acid sequence having at least 85% sequence identity with SEQ ID NO:2, and an amino acid sequence having at least 80% sequence identity with is SEQ ID NO:2 and at least 50% of the activity thereof; and
b) detecting the presence and/or absence of binding between said compound and said polypeptide;
wherein binding indicates that said compound is a candidate for a herbicide.

7. A method for determining whether a compound identified as a herbicide candidate by the method of claim 6 has herbicidal activity, comprising: contacting a plant or plant cells with said herbicide candidate and detecting the presence or absence of a decrease in growth or viability of said plant or plant cells.

8. A method for identifying a compound as a candidate for a herbicide, comprising:
a) contacting a vestitone and NADP+ with a plant IFR;
a) contacting said vestitone and NADP+ with IFR and said candidate compound;
c) determining the concentration of at least one of vestitone, NADP+, 2′-hydroxyformononetin, and/or NADPH after the contacting of steps (a) and (b).

9. The method of claim 8, wherein said IFR is an Arabidopsis IFR.

10. The method of claim 9, wherein said IFR is SEQ ID NO:2.

11. The method of claim 10, wherein said IFR consists essentially of SEQ ID NO:2.

12. A method for identifying a compound as a candidate for a herbicide, comprising:
a) contacting a 2′-hydroxyformononetin and NADPH with a plant IFR;
b) contacting said 2′-hydroxyformononetin and NADPH with said IFR and said candidate compound; and
c) determining the concentration of at least one of vestitone, NADP+, 2′-hydroxyformononetin, and/or NADPH after the contacting of steps (a) and (b).

13. The method of claim 12, wherein said IFR is an Arabidopsis IFR.

14. The method of claim 12, wherein said IFR is SEQ ID NO:2.

15. The method of claim 12, wherein said IFR consists essentially SEQ ID NO:2.

16. A method for identifying a compound as a candidate for a herbicide, comprising:
a) contacting vestitone and NADP+ with a polypeptide selected from the group consisting of: a polypeptide having at least 85% sequence identity with SEQ ID NO:2, a polypeptide having at least 80% sequence identity with SEQ ID NO:2 and at least 50% of the activity thereof, and a polypeptide comprising at least 100 consecutive amino acids of SEQ ID NO:2;
b) contacting said vestitone and NADP+ with said polypeptide and said compound;
c) determining the concentration of at least one of vestitone, NADP+, 2′-hydroxyformononetin and/or NADPH after the contacting of steps (a) and (b).

17. A method for identifying a compound as a candidate for a herbicide, comprising:
a) contacting a 2′-hydroxyformononetin and NADPH with a polypeptide selected from the group consisting of: a polypeptide having at least 85% sequence identity with SEQ ID NO:2, a polypeptide having at least 80% sequence identity with SEQ ID NO:2 and at least 50% of the activity thereof, and a polypeptide comprising at least 100 consecutive amino acids of SEQ ID NO:2;
b) contacting said 2′-hydroxyformononetin and NADPH + with said polypeptide and said compound; and
c) determining the concentration of at least one of vestitone, NADP+, 2′-hydroxyformononetin and/or NADPH after the contacting of steps (a) and (b).

18. A method for identifying a compound as a candidate for a herbicide, comprising:
a) measuring the expression of an IFR in a plant or plant cell in the absence of said compound;
b) contacting a plant or plant cell with said compound and measuring the expression of said IFR in said plant or plant cell;
c) comparing the expression of IFR in steps (a) and (b).

19. The method of claim 13 wherein said plant or plant cell is an Arabidopsis plant or plant cell.

20. The method of claim 14, wherein said IFR is SEQ ID NO 2.

21. The method of claim 13, wherein the expression of IFR is measured by detecting IFR mRNA.

22. The method of claim 13, wherein the expression of IFR is measured by detecting IFR polypeptide.
